(19) United States
(12) Patent Application Publication
(10) Pub. No.: US 2024/0324949 A1
(43) Pub. Date: Oct. 3, 2024

(54) SLEEP DISRUPTIONS IDENTIFICATION FROM MILLIMETER-WAVE WIRELESS SYSTEMS

(71) Applicant: UNIVERSITY OF SOUTH CAROLINA, COLUMBIA, SC (US)

(72) Inventor: SANJIB SUR, CAYCE, SC (US)

(21) Appl. No.: 18/429,626

(22) Filed: Feb. 1, 2024

Related U.S. Application Data
(60) Provisional application No. 63/493,864, filed on Apr. 3, 2023.

(51) Int. Cl. A61B 5/00; A61B 5/11; A61B 90/00; G16H 50/70

(52) U.S. Cl. CPC A61B 5/4815; A61B 5/002; A61B 5/1116; A61B 5/1126; A61B 5/4809; A61B 5/4812; A61B 5/7225; A61B 5/7239; A61B 5/7246; A61B 5/7257; A61B 5/7267; A61B 90/06; G16H 50/70

(57) ABSTRACT

Methodology and corresponding apparatus pertains to sleep disruption monitoring, including use of a wireless signal-based monitoring system leveraging millimeter-wave technology. A software-only sleep disruption monitoring solution can be based on millimeter-wave (mmWave) wireless-based solutions which leverage cross-correlation between successive mmWave reflected signals and a Hidden Markov Model (HMM) to identify respective sleep (rest) and disruptions (toss-turn) periods. A toss-turn detector module can identify sudden movements during sleep from mmWave wireless signals and classify the sleeping period into the two states: Rest or toss-turn. Whenever mmWave transceivers (such as included in 5G-and-beyond devices) are implemented as access points, in mass privacy non-invasive sleep disruption monitoring can be provided for consumers at home.

### PRIORITY CLAIM

The present application claims the benefit of priority of U.S. Provisional Patent Application No. 63/493,864, filed Apr. 3, 2023, which is titled Sleep Disruptions Identification From Millimeter-wave Wireless Systems, and which is fully incorporated herein by reference for all purposes.

### STATEMENT REGARDING SPONSORED RESEARCH OR DEVELOPMENT

This invention was made with government support under Grant Number 2144505, awarded by the NSF. The government has certain rights in the invention.

### BACKGROUND OF THE PRESENTLY DISCLOSED SUBJECT MATTER

The presently disclosed subject matter generally relates to sleep disruption monitoring, and more particularly to a wireless signal-based sleep disruption monitoring system.

Sleep is a critical aspect of human health and well-being, particularly for children's neurological development and adult wellbeing. Existing works using parent reports show that disrupted sleep in early infancy is related to atypical neurodevelopment. Therefore, studying sleep disruptions, such as toss-turn patterns, can provide insight into the impact of sleep on the overall functioning of adults and future cognitive development of infants.

However, such general methodology has severe limitations. For example, it is incredibly physically invasive, complicated to set up in-home, and, critically, can disrupt the infant's natural sleeping pattern and often lead to rashes, burns, and injuries.

In view of the foregoing, there is a significant need for non-invasive and contactless approaches that estimate sleep disruptions without compromising the comfort and safety of the subjects or patients.

The presently disclosed technology would make it possible to address such shortcomings. In particular, millimeter-wave (mmWave) wireless-based solutions presently disclosed can overcome such challenges by generally enabling fine-grained disruption monitoring.

### SUMMARY OF THE PRESENTLY DISCLOSED SUBJECT MATTER

The presently disclosed technology (systems and/or corresponding and/or associated methodology) generally relates to sleep disruption monitoring. Such subject matter more particularly relates to a wireless signal-based sleep disruption monitoring system.

For some embodiments, the presently disclosed subject matter leverages built-in millimeter-wave technology on ubiquitous 5G wireless devices and discloses a software-only sleep disruption monitoring solution. For such embodiments, advantageously the presently disclosed subject matter does not require any additional hardware such as used with existing pressure-sensing mattresses.

Presently disclosed millimeter-wave (mmWave) wireless-based solutions can overcome existing challenges in this area of technology (sleep disruption identification) by enabling fine-grained disruption monitoring from wireless signal reflections. The presently disclosed technology facilitates the ability to leverage cross-correlation between successive mmWave reflected signals and a Hidden Markov Model (HMM) to identify the sleep and disruptions period.

Additionally, the presently disclosed technology is particularly positioned to take advantage of mmWave transceivers, which are poised to soon become ubiquitous in all 5G-and-beyond devices, such as access points. Therefore, certain embodiments of the presently disclosed technology can be potentially enabled through available devices. Such fact results in the opportunity for bringing privacy non-invasive sleep disruption monitoring to the masses at-home.

It is to be understood that the presently disclosed subject matter equally relates to associated and/or corresponding methodologies and operative devices or technologies. One exemplary such method relates to methodology for identifying sleep disruptions of a human subject, comprising transmitting millimeter-wave (mmWave) wireless signals configured for interacting with a human subject; receiving millimeter-wave (mmWave) wireless signals reflecting from the human subject; identifying movements of the human subject based on the received signal reflections; and based on identified movements, classifying the posture of the human subject into one of two states of rest or toss-turn.

Another exemplary such method relates to a method for automatically identifying sleep disruptions of a human subject from millimeter-wave (mmWave) wireless signals reflecting from the human subject. Such method preferably comprises training a two-states Hidden Markov Model (HMM)-based rest and toss-turn detection machine learning model, based on inputs of ground truth rest or toss-turn states of a plurality of human subjects and corresponding generated input-output pairs of mmWave reflected signals from the plurality of human subjects, to learn the association between millimeter-wave (mmWave) wireless signals reflected from a human subject and rest and toss-turn states of a human subject; and operating the trained rest and toss-turn detection machine learning model to process further input data thereto, to determine and output identification of rest and toss-turn states of a human subject.

Other example aspects of the present disclosure are directed to systems, apparatus, tangible, non-transitory computer-readable media, user interfaces, memory devices, and electronic devices for mmWave imaging. To implement methodology and technology herewith, one or more processors may be provided, programmed to perform the steps and functions as called for by the presently disclosed subject matter, as will be understood by those of ordinary skill in the art.

Another exemplary embodiment of presently disclosed subject matter relates to a one or more tangible, non-transitory computer-readable media that collectively store instructions that, when executed, cause a computing device including one or more processors to perform operations, the operations comprising automatically identifying sleep disruptions of a human subject from millimeter-wave (mmWave) wireless signals reflecting from the human subject, by training a two-states Hidden Markov Model (HMM)-based rest and toss-turn detection machine learning model, based on inputs of ground truth rest or toss-turn states of a plurality of human subjects and corresponding generated input-output pairs of mmWave reflected signals from the plurality of human subjects, to learn the association between millimeter-wave (mmWave) wireless signals reflected from a human subject and rest and toss-turn states of a human subject; and operating the trained rest and toss-turn detection machine learning model to process further input data thereto, to determine and output identification of rest and toss-turn states of a human subject.

Additional objects and advantages of the presently disclosed subject matter are set forth in, or will be apparent to, those of ordinary skill in the art from the detailed description herein. Also, it should be further appreciated that modifications and variations to the specifically illustrated, referred and discussed features, elements, and steps hereof may be practiced in various embodiments, uses, and practices of the presently disclosed subject matter without departing from the spirit and scope of the subject matter. Variations may include, but are not limited to, substitution of equivalent means, features, or steps for those illustrated, referenced, or discussed, and the functional, operational, or positional reversal of various parts, features, steps, or the like.

Still further, it is to be understood that different embodiments, as well as different presently preferred embodiments, of the presently disclosed subject matter may include various combinations or configurations of presently disclosed features, steps, or elements, or their equivalents (including combinations of features, parts, or steps or configurations thereof not expressly shown in the figures or stated in the detailed description of such figures). Additional embodiments of the presently disclosed subject matter, not necessarily expressed in the summarized section, may include and incorporate various combinations of aspects of features, components, or steps referenced in the summarized objects above, and/or other features, components, or steps as otherwise discussed in this application. Those of ordinary skill in the art will better appreciate the features and aspects of such embodiments, and others, upon review of the remainder of the specification, and will appreciate that the presently disclosed subject matter applies equally to corresponding methodologies as associated with practice of any of the present exemplary devices, and vice versa.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1(a) illustrates an exemplary arrangement or set-up in accordance with presently disclosed subject matter whereby a mmWave device receives reflected signals from a human (such as a sleeping subject or patient);

FIG. 1(b) graphically illustrates an example of reflected signals as may be received from a person (subject) in accordance with the presently disclosed subject matter, such as in the arrangement represented in present FIG. 1(a);

FIGS. 2(a) and 2(b) graphically illustrate respectively Short-Time Fourier Transform (STFT) outputs from two exemplary monitored cases, such as in the arrangement represented in present FIG. 1(a), with exemplary three and four toss-turn events, respectively;

FIG. 3 graphically represents determination of exemplary toss-turn/rest states detected in accordance with presently disclosed subject matter, including illustrating cross-correlation between reflected mmWave signals, time-derivative representations of such signals, and envelope estimations with Root-Mean-Square (RMS) of samples for 1 second;

FIG. 4 graphically represents monitoring and determining of exemplary toss-turn/rest states detected in accordance with presently disclosed subject matter, including illustrating presently disclosed envelope detector output from mmWave signals shown, for example, as following posture changes;

FIG. 5(a) diagrammatically represents a two-states Hidden Markov Model (HMM) to identify the respective sleep (“rest”) and disruptions (“toss-turn”) periods;

FIG. 5(b) graphically illustrates an example of a period of time of monitoring for a plurality of rest and toss-turn states/events in accordance with presently disclosed subject matter, and comparing predictions with a ground truth based output;

FIG. 6(a) graphically illustrates effects on rest and toss-turn detection accuracy of presently disclosed subject matter based on varying the number of antennas (for detection);

FIG. 6(b) graphically illustrates effects on rest and toss-turn detection accuracy of presently disclosed subject matter based on using different RMS durations for envelope estimation;

FIG. 7(a) graphically illustrates precision, recall, and F1-score for the presently disclosed state detection using Hidden Markov Model (HMM) based methodology;

FIG. 7(b) graphically illustrates distribution of errors in toss-turn duration estimation, with and without presently disclosed state detection using Hidden Markov Model (HMM) based methodology; and

FIG. 7(c) graphically illustrates distribution of errors in toss-turn start times and end times detections using Hidden Markov Model (HMM) based methodology.

Repeat use of reference characters in the present specification and drawings is intended to represent the same or analogous features, elements, or steps of the presently disclosed subject matter.

### DETAILED DESCRIPTION OF THE PRESENTLY DISCLOSED SUBJECT MATTER

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

### 1. General Approach

In general, the present disclosure is directed to technology which generally relates to sleep disruption monitoring. The presently disclosed subject matter more particularly relates to a wireless signal-based sleep disruption monitoring system.

For some embodiments, the presently disclosed subject matter can leverage built-in millimeter-wave technology on ubiquitous 5G wireless devices. Therefore, in some instances, the presently disclosed subject matter discloses a software-only sleep disruption monitoring solution. For such embodiments, advantageously the presently disclosed subject matter does not require any additional hardware such as used with existing pressure-sensing mattresses.

One of the significant aspects of some of the presently disclosed millimeter-wave (mmWave) wireless-based solutions is to leverage cross-correlation between successive mmWave reflected signals and a Hidden Markov Model (HMM) to identify the sleep and disruptions period. Whenever mmWave transceivers (such as included in 5G-and-beyond devices) are implemented as access points, the presently disclosed technology makes it possible to provide in mass privacy non-invasive sleep disruption monitoring for consumers at home.

### 2. Disclosed Approach

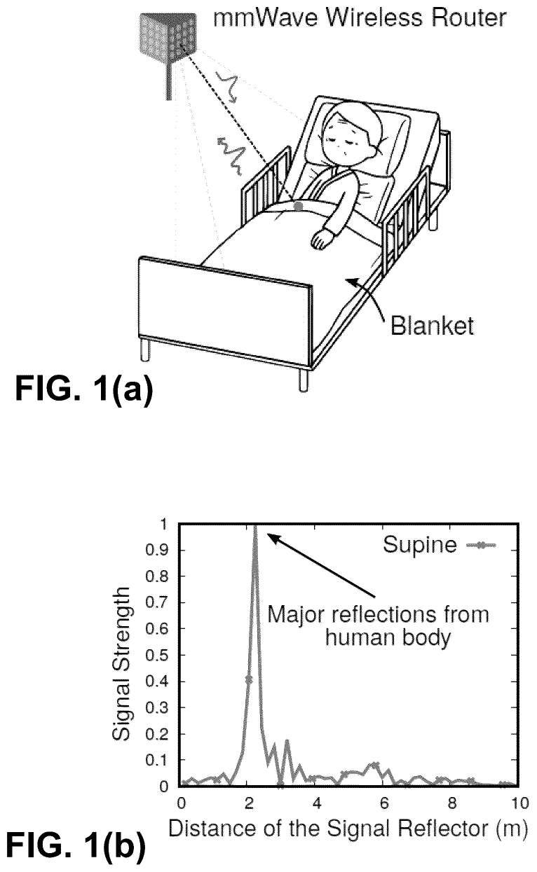

The core purpose of the toss-turn detector module is to identify sudden movements during sleep from mmWave wireless signals and classify the sleeping period into two states: Rest or toss-turn. FIGS. 1(a) and 1(b) show an example of mmWave signal reflections from a sleeping human (such as a subject, study subject, or patient). In particular, FIG. 1(a) illustrates an exemplary arrangement or set-up in accordance with presently disclosed subject matter whereby a mmWave device receives reflected signals from a human (such as a sleeping subject or patient). Also, FIG. 1(b) graphically illustrates an example of reflected signals as may be received from a person (subject) in accordance with the presently disclosed subject matter, such as in the arrangement represented in present FIG. 1(a).

It is critical to identify and separate the two respective states (rest or toss-turn) as it not only helps in estimating the time gap between two adjacent resting periods but also facilitates a deep learning model to only predict the posture under rest states, and avoid erroneous predictions under toss-turns.

### Cross-Correlation Based Toss-Turn Detection:

Previous work has related to elderly fall detection using wireless signals [1]. Implementations and aspects of the presently disclosed subject matter is in some instances alternatively referred to as “ArgoSleep.” ArgoSleep leverages the observation that in comparison to the rest states, toss-turn states are usually associated with significantly higher spatio-temporal changes in the received mmWave signals.

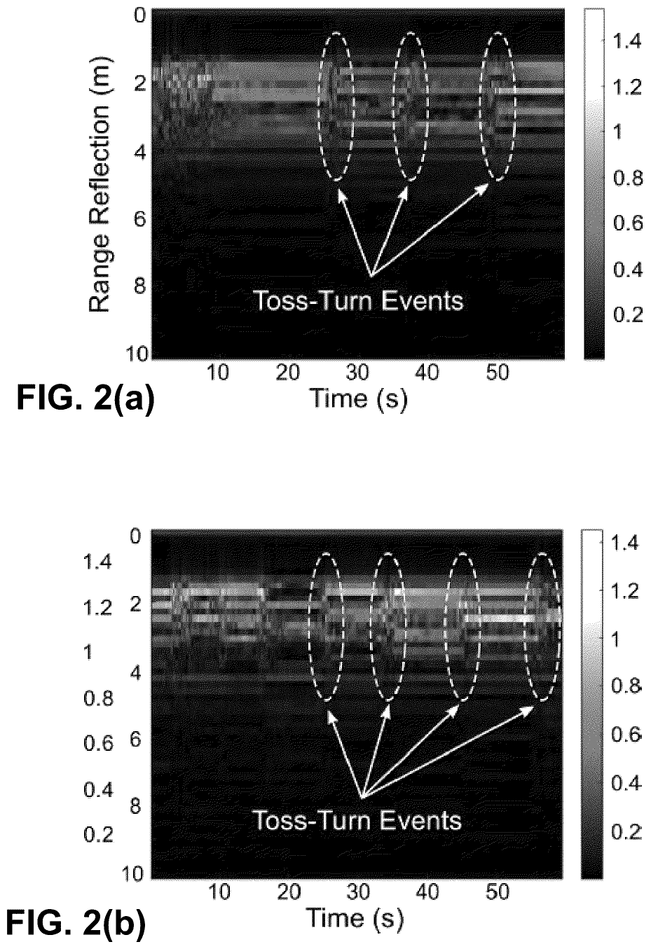

To observe distinct toss-turn states, a volunteer was asked to lie down on the bed placed at 2.5 m in front of the mmWave devices for about 60 seconds and perform multiple toss-turns, i.e., move from one posture to another. A Short-Time Fourier Transform (STFT) was performed over the signal received by an exemplary one of the mmWave antennas from a test set-up. FIGS. 2(a) and 2(b) reflect results from such set-up. In particular, FIGS. 2(a) and 2(b) graphically illustrate respectively Short-Time Fourier Transform (STFT) outputs from two exemplary monitored cases, such as in the arrangement represented in present FIG. 1(a), with exemplary three and four toss-turn events, respectively.

In comparison for example to falls, a toss-turn event during sleep is usually a relatively small-scale event. For example, the centroid of the body might not change, while the limbs typically move between adjacent distance m (FIG. 1(b)) of the reflected signals. The changes observed under the toss-turn are accordingly much weaker and do not show stark time-and-frequency changes. Therefore, additional processing is required to amplify the changes during the toss-turns and separate them from the rest states.

To amplify such changes, ArgoSleep applies a cross-correlation between successive frames of the reflected signals, and estimates the rate of change (i.e., time-derivative) in the peak correlation output. The key idea is intuitive: Cross-correlation between successive frames allows uncovering the similarity (or dissimilarity) between the consecutive reflected signals. Since during the rest states, there are almost no changes in the successive reflected signals, the cross-correlation will show almost the same peak; so, its rate of change over time should be close to zero. On the other hand, during the toss-turn states, such correlation peak fluctuates significantly, with a variable rate of change. More importantly, the time-derivative removes the almost constant reflections from the static background, i.e., bed, furniture, nightstands, etc., so that the only changes due to the body movement are amplified and stand out. Let's consider, Rₜ ({d₁, d₂, . . . , dₙ,}) as the reflected signal at time t from distances dₜ w.r.t. the receiver.

Mathematically, the cross-correlation, xCorr can be expressed as

$$x {Corr}_{t} = {max}_{m ∈ ( 0 , n - 1 )} \vert {\sum }_{i = 1}^{n - m} {R}_{t} ( {d}_{i + m} ) \cdot {R}_{t - 1}^{*} ( {d}_{i} ) \vert$$

and its time-derivative, ΔxCorr can be expressed as

$$Δ x {Corr}_{t - 1}^{t} = x {Corr}_{t} - x {Corr}_{t - 1} ,$$

where Rₜ₋₁* is the complex conjugate of the received reflected signal at time instant t-1.

To reduce the number of oscillations between the false detections (+/−) and states, ArgoSleep smoothens ΔxCorr over time with an envelope detector using a Hilbert Transformation, similar to [2]. The envelope detector uses the Root-Mean-Square (RMS) of ΔxCorr amplitudes over N consecutive frames. Intuitively, a large value of N suppresses many false detections but will have a slow reaction to the true state change. On the other hand, a small value of N will have a fast reaction but could lead to high false detections and state oscillations. In practice, N=25 frames, i.e., 1 second of the consecutive reflected signals, for envelope estimation, yields a good result, since human movements during the sleep are on the order of several seconds.

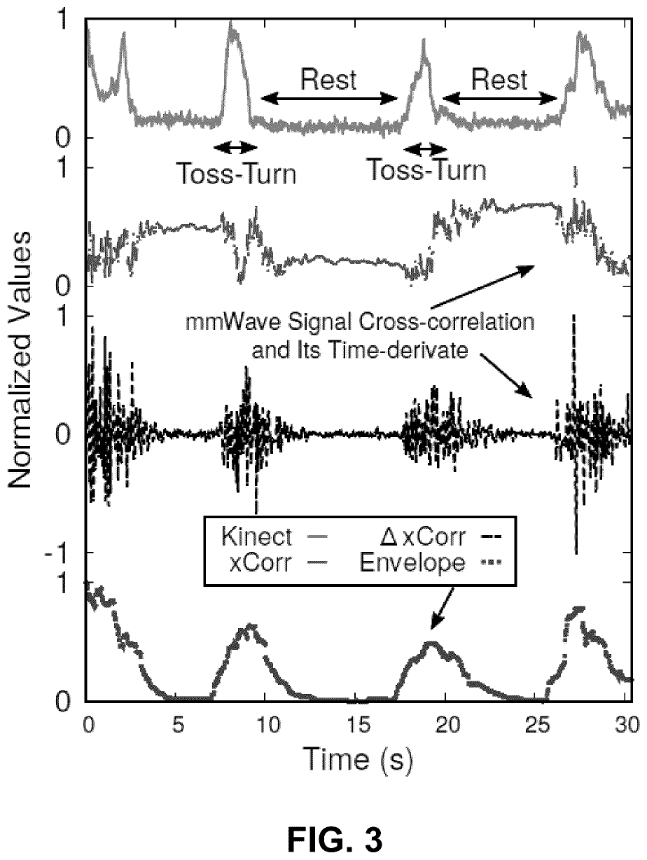

FIG. 3 shows an example cross-correlation result. In particular, FIG. 3 graphically represents determination of exemplary toss-turn/rest states detected in accordance with presently disclosed subject matter, including illustrating cross-correlation between reflected mmWave signals, time-derivative representations of such signals, and envelope estimations with Root-Mean-Square (RMS) of samples for 1 second.

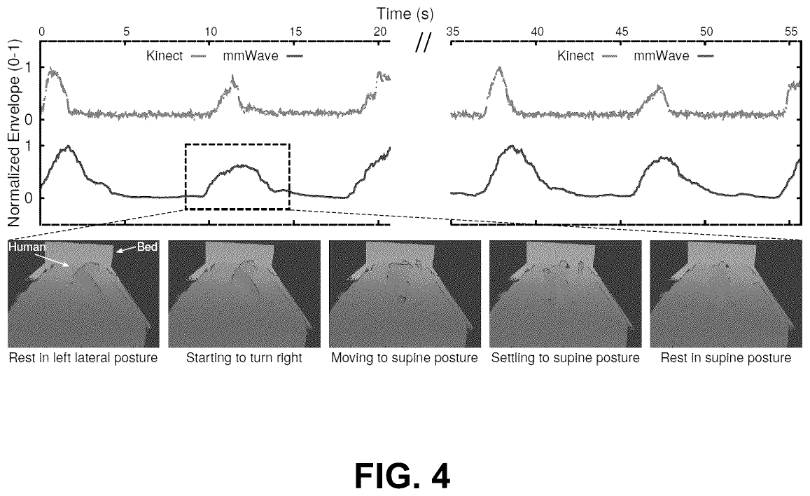

FIG. 4 shows another output of the envelope detector, and compares the result with the Kinect based output (from a camera-based Kinect sensor, originally developed for video game play). In particular, FIG. 4 graphically represents monitoring and determining of exemplary toss-turn/rest states detected in accordance with presently disclosed subject matter, including illustrating presently disclosed envelope detector output from mmWave signals shown, for example, as following posture changes. In other words, the envelope detector output from mmWave signals follows the posture changes.

FIG. 4 also shows a zoomed time period, where a volunteer in the left lateral posture turns right and moves to the supine posture. However, using the envelope output for state detection is challenging as the output of the envelope detector is a real number between 0 to 1, where 0 indicates no change in the successive reflected signals, and 1 indicates a very high change. But for posture detection, ArgoSleep requires discrete binary states: Rest or toss-turn. Further, due to sensitivity of mmWave signals to minute changes in the environment, the envelope detector can still show high output during the occasional hand or leg movements, even if the full body has not turned yet. Also, there could be early toss start and late toss end detection, leading to the wrong estimation of the event duration: See FIG. 4, where the turn time duration estimated by mmWave signals is much larger than the Kinect output.

Improving Detection with a Two-States HMM:

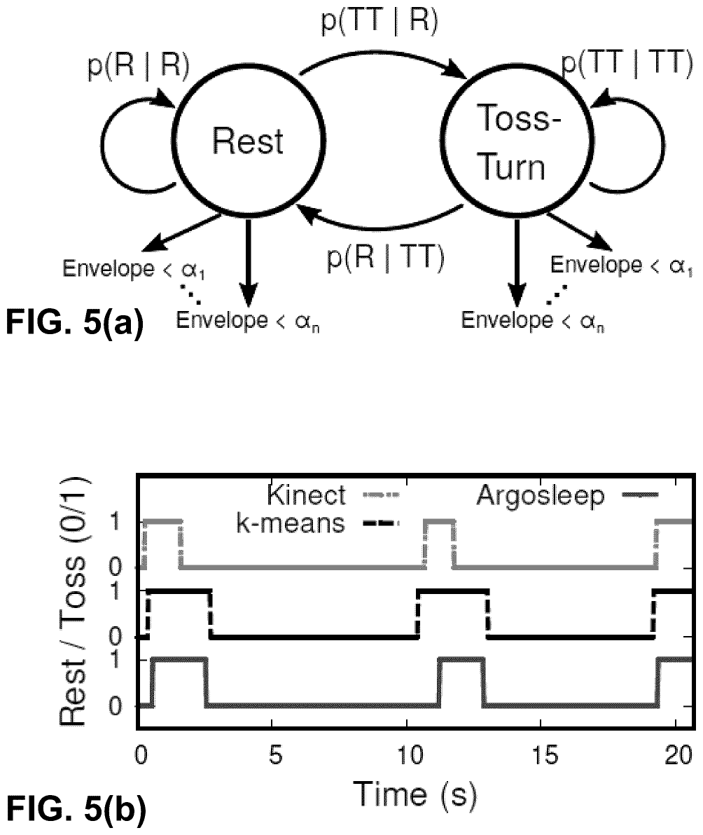

To overcome this challenge and improve the detection accuracy and timing estimations, we disclose an exemplary lightweight two-states HMM [3]. The HMM not only converts the envelope with real-valued output between 0 to 1 to a discrete output of 0 and 1, but also improves the state detection accuracy and reduces the timing errors. FIG. 5(a) diagrammatically represents a two-states Hidden Markov Model (HMM) to identify the respective sleep (“rest”) and disruptions (“toss-turn”) periods. Thus, FIG. 5(a) shows the state transition diagram of ArgoSleep's HMM: with the two states being rest and toss-turn, and the emissions being different levels of envelope values. To build the HMM, we collect several ground truth datasets involving multiple volunteers tossing and turning during their sleep, and formulate the state transition matrices by estimating the 4 conditional probabilities, i.e.,

p(Rest|Rest),p(Rest|Toss-Turn),p(Toss-Turn|Rest), and p(Toss-Turn|Toss-Turn).

We formulate the emission matrix by estimating the conditional probabilities for discrete envelope values (e), i.e.,

p(e<α₁|Rest),p(e<α₂|Rest), . . . ,p(e<β₁|Toss-Turn),p(e<α₂|Toss-Turn).

and so on.

Finally, at run-time, ArgoSleep first calculates the envelope from the reflected mmWave signals, and then uses the state transition and emission matrices and a Viterbi decoder to predict the binary states, corresponding to rest and toss-turn. FIG. 5(b) graphically illustrates an example of a period of time of monitoring for a plurality of rest and toss-turn states/events in accordance with presently disclosed subject matter, and comparing predictions with a ground truth based output (which in this case is a Kinect-based output). FIG. 5(b) in particular shows an example of about 20 seconds of monitoring with three toss-turn events and compares the prediction with the ground truth Kinect based output. Clearly, in comparison to the k-means with adaptive threshold, HMM can improve the errors in event start and stop times. Once the entire sleeping period is classified into either states, ArgoSleep aims to predict the sleep posture during the rest state.

### 2.1. Results

### State Detection Performance:

We first evaluate the effectiveness of ArgoSleep's toss-turn detection modules. Since it is hard to control the number of toss-turns during the actual sleep, and obtain a reasonably-sized dataset, we obtain 19,386 state observations from 30 datasets collected from a volunteer who mimics the toss-turn events with posture changes within a short period of 20 to 30 seconds to generate one dataset. We generate the input-output pairs of mmWave reflected signals and 3D location of body joints and also, corresponding Kinect depth images to identify the ground truth rest or toss-turn states. We find the ground truth toss-turn by applying a fixed mask and calculating the pixel-to-pixel difference in successive depth images and then, finding the energy in residual depth. Then, we build the HMM from the Kinect ground truth, use the reflected signals to estimate the envelope, and apply the Viterbi decoder to it to predict the states.

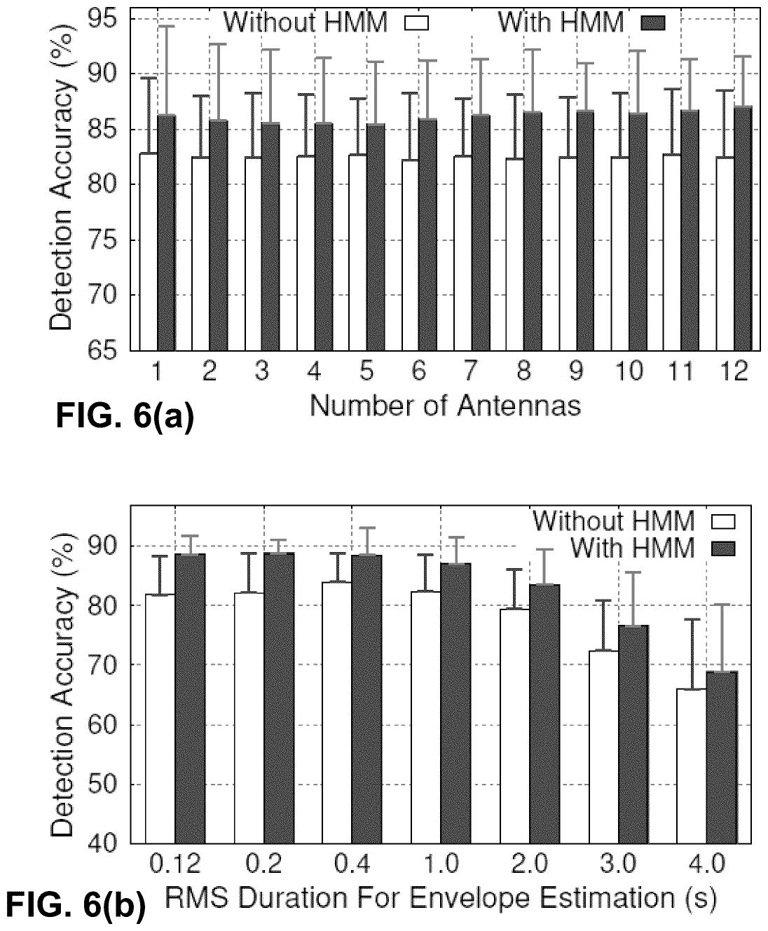

FIGS. 6(a) and 6(b) relate to rest and toss-turn detection accuracy using the presently disclosed technology. FIG. 6(a) shows the state detection accuracy with respect to the Kinect-based ground truth, across different number of antennas. In other words, FIG. 6(a) graphically illustrates effects on rest and toss-turn detection accuracy of presently disclosed subject matter based on varying the number of antennas (for detection). In particular, the graphically represented bar and errorbar features represent the median and standard deviation data, respectively, across 19,386 exemplary states.

We use the observation from multiple antennas and take median votes to decide between the output binary states. Clearly, ArgoSleep's HMM-Viterbi performs consistently better than the envelope thresholding algorithm, and the median accuracy is always above 85%, reaching up to 100% in certain cases. This is because the HMM-Viterbi can enforce the envelope to follow the Kinect based toss-turn events with its state and emission matrices. Moreover, the detection accuracy is unaffected by the number of antennas since a single antenna, with a large beamwidth, can cover the whole bed area. We also use a variable number of frames, from 3 to 100, corresponding to 0.12 to 4 s, to compute the RMS for the envelope detector, and predict the states.

FIG. 6(b) graphically illustrates effects on rest and toss-turn detection accuracy of presently disclosed subject matter based on using different Root-Mean-Square (RMS) durations for envelope estimation. FIG. 6(b) shows that the large RMS duration, such as 4 s, although useful for suppressing false detections, decreases the state detection accuracy significantly, since it reacts slowly to the true state changes. Still, ArgoSleep performs consistently better with HMM-Viterbi, and RMS duration of 1 s shows 88% detection accuracy on the median.

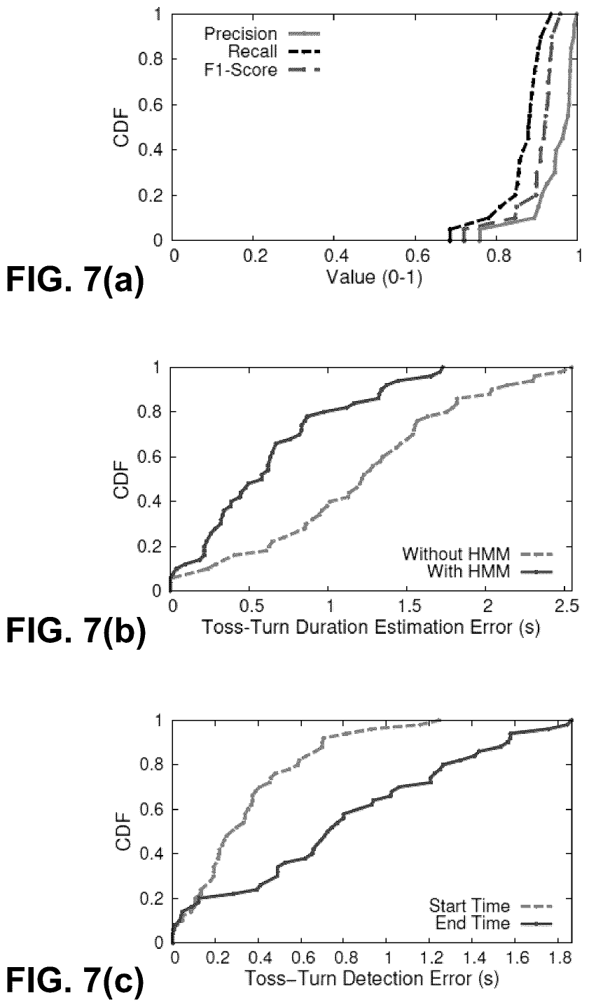

FIG. 7(a) graphically illustrates precision, recall, and F1-score for the presently disclosed state detection using Hidden Markov Model (HMM) based methodology. In other words, FIG. 7(a) shows the distribution of precision, recall, and F1-score of the event detection, where the median values are 0.97, 0.88, and 0.92, respectively, indicating ArgoSleep is not only accurate but also has low false detection rates.

### Toss-Turn Timing Parameters:

Next, we evaluate ArgoSleep's performance in identifying the timing of the toss-turn events. This information could be useful in not only identifying the precise start and end of toss-turn but also annotating the events automatically. To this end, we use the same set of state observations as before and estimate the toss-turn times from both ArgoSleep and ground truth. We evaluate three different errors in timing parameters: Toss-turn start time, end time, and duration. For the start and end times, we first locate each event in the ground truth and identify the time of the state change from 0 to 1 (i.e., rest to toss-turn) as start and 1 to 0 (i.e., toss-turn to rest) as an end. For each case, we identify the closest time of such events detected by ArgoSleep and estimate their corresponding start and end times. For the duration error, we find the sum of the absolute differences in start and end times from the ground truth and ArgoSleep.

FIG. 7(b) graphically illustrates distribution of errors in toss-turn duration estimation, with and without presently disclosed state detection using Hidden Markov Model (HMM) based methodology. In particular, FIG. 7(b) shows the distribution of error in duration estimation across about 100 toss-turn events, and compares the performance with and without the HMM-Viterbi. First, our total count for predicted toss-turn events shows that ArgoSleep did not miss any detection. Second, without HMM-Viterbi, we observe that the median and 90th percentile errors are 1.22 s and 2.04 s, respectively. In contrast, HMM-Viterbi can reduce this error to 0.58 s and 1.34 s in median and 90th percentile, respectively. More importantly, ArgoSleep always predicts the duration within 1.7 s of the ground truth across all our observed events.

FIG. 7(c) further shows the toss-turn start and end time estimation errors with presently disclosed HMM-Viterbi technology. In particular, FIG. 7(c) graphically illustrates distribution of errors in toss-turn start times and end times detections using Hidden Markov Model (HMM) based methodology. Here, the median errors in start and end detections are 0.25 s and 0.73 s, respectively. Moreover, the 90th percentile errors show that ArgoSleep is accurate within 1 s and 1.5 s to detect the start and end of the event, respectively.

This written description uses examples to disclose the presently disclosed subject matter, including the best mode, and also to enable any person skilled in the art to practice the presently disclosed subject matter, including making and using any devices or systems and performing any incorporated methods. The patentable scope of the presently disclosed subject matter is defined by the claims, and may include other examples that occur to those skilled in the art. Such other examples are intended to be within the scope of the claims if they include structural and/or step elements that do not differ from the literal language of the claims, or if they include equivalent structural and/or elements with insubstantial differences from the literal languages of the claims. In any event, while certain embodiments of the disclosed subject matter have been described using specific terms, such description is for illustrative purposes only, and it is to be understood that changes and variations may be made without departing from the spirit or scope of the subject matter. Also, for purposes of the present disclosure, the terms “a” or “an” entity or object refers to one or more of such entity or object. Accordingly, the terms “a”, “an”, “one or more,” and “at least one” can be used interchangeably herein.

### REFERENCES

[1] Wang, Xueyi and Ellul, Joshua and Azzopardi, George, “Elderly Fall Detection Systems: A Literature Survey,” Frontiers in Robotics and AI, vol. 7, 2020.
[2] Yisak Kim and Juyoung Park and Hyungsuk Kim, “Signal-Processing Framework for Ultrasound Compressed Sensing Data: Envelope Detection and Spectral Analysis,” in MDPI Applied Sciences, 2020.
[3] Daniel Jurafsky and James H. Martin, Speech and Language Processing, 3rd ed. Pearson Prentice Hall, 2021.

What is claimed is:

1. Methodology for identifying sleep disruptions of a human subject, comprising:
transmitting millimeter-wave (mmWave) wireless signals configured for interacting with a human subject;
receiving millimeter-wave (mmWave) wireless signals reflecting from the human subject;
identifying movements of the human subject based on the received signal reflections; and
based on identified movements, classifying the posture of the human subject into one of two states of rest or toss-turn.

2. Methodology according to claim 1, further comprising:
determining temporal estimations of the beginning and end of the respective states, and length of states, during a time period during which a human subject is monitored for a plurality of rest and toss-turn states.

3. Methodology according to claim 1, wherein identifying movements includes processing the received signal reflections to amplify toss-turn changes to distinguish them from rest states.

4. Methodology according to claim 3, further comprising performing Short-Time Fourier Transform (STFT) processing on the received signal reflections.

5. Methodology according to claim 3, further comprising:
amplifying toss-turn changes by applying cross-correlation between successive frames of the reflected signals; and
estimating the rate of change in the peak correlation output.

6. Methodology according to claim 5, wherein the estimating comprises using the time-derivatives of the reflected signal cross-correlations.

7. Methodology according to claim 6, further comprising, to reduce oscillations between false detections and states, smoothing the cross-correlations over time by using an envelope detector.

8. Methodology according to claim 7, wherein using the envelope detector comprises using a Hilbert Transformation, using the Root-Mean-Square (RMS) of cross-correlation amplitudes over N consecutive frames.

9. Methodology according to claim 8, wherein the number of N consecutive frames is about 25, for an RMS resolution of about 1 second of consecutive reflected signals, for envelope estimation.

10. Methodology according to claim 8, wherein the number of N consecutive frames is in a range of about 3 to 50, for a corresponding range of RMS resolution of about 0.12 to 2 seconds of consecutive reflected signals, for envelope estimation.

11. Methodology according to claim 1, further comprising determining:
cross-correlations between consecutive reflected mmWave signals,
time-derivative representations of the cross-correlations, and
envelope estimations of the time-derivative representations with Root-Mean-Square (RMS) of samples for about one second, and
posture classifying based on the envelope estimations.

12. Methodology according to claim 2, further comprising monitoring a human subject using an observation arrangement in which a human subject is reclined on a bed, and at least one mmWave transmitter and receiving antenna is positioned in a range from 2 to 5 meters away from the human subject, with the antenna having a sufficiently large beamwidth to cover the whole bed area of the bed on which the human subject is reclined.

13. Method for automatically identifying sleep disruptions of a human subject from millimeter-wave (mmWave) wireless signals reflecting from the human subject, comprising:
training a two-states Hidden Markov Model (HMM)-based rest and toss-turn detection machine learning model, based on inputs of ground truth rest or toss-turn states of a plurality of human subjects and corresponding generated input-output pairs of mmWave reflected signals from the plurality of human subjects, to learn the association between millimeter-wave (mmWave) wireless signals reflected from a human subject and rest and toss-turn states of a human subject; and
operating the trained rest and toss-turn detection machine learning model to process further input data thereto, to determine and output identification of rest and toss-turn states of a human subject.

14. The method according to claim 13, wherein the inputs of ground truth rest or toss-turn states of the plurality of human subjects are based on corresponding depth images of the plurality of human subjects to identify the ground truth rest or toss-turn states.

15. The method according to claim 14, wherein the ground truth toss-turn is found by applying a fixed mask to the depth images and calculating the pixel-to-pixel difference in successive depth images, and then finding the energy in residual depth.

16. The method according to claim 13, wherein training includes leveraging cross-correlations between successive mmWave reflected signals to identify respective sleep versus disruption periods.

17. The method according to claim 13, wherein the rest and toss-turn detection machine learning model is further trained to identify and separate two respective states of rest or toss-turn, and to estimate time gap between two adjacent resting periods.

18. The method according to claim 13, wherein training includes calculating envelopes from the reflected mmWave signals, and then predicting the binary states corresponding to rest and toss-turn.

19. The method according to claim 18, further comprising converting the calculated envelopes with real-valued output between 0 to 1 to a discrete output of 0 and 1 as the predicted binary states corresponding to rest and toss-turn.

20. The method according to claim 18, wherein predicting further comprises using state transition and emission matrices and a Viterbi decoder to predict the binary states, corresponding to rest and toss-turn.

21. The method according to claim 18, wherein calculating envelopes comprises using a Hilbert Transformation, using the Root-Mean-Square (RMS) of cross-correlation amplitudes over N consecutive frames, wherein the number of N consecutive frames is in a range of about 3 to 50, for a corresponding range of RMS resolution of about 0.12 to 2 seconds of consecutive reflected signals, for envelope calculation.

22. One or more tangible, non-transitory computer-readable media that collectively store instructions that, when executed, cause a computing device including one or more processors to perform operations, the operations comprising automatically identifying sleep disruptions of a human subject from millimeter-wave (mmWave) wireless signals reflecting from the human subject, by:
training a two-states Hidden Markov Model (HMM)-based rest and toss-turn detection machine learning model, based on inputs of ground truth rest or toss-turn states of a plurality of human subjects and corresponding generated input-output pairs of mmWave reflected signals from the plurality of human subjects, to learn the association between millimeter-wave (mmWave) wireless signals reflected from a human subject and rest and toss-turn states of a human subject; and
operating the trained rest and toss-turn detection machine learning model to process further input data thereto, to determine and output identification of rest and toss-turn states of a human subject.

23. The one or more tangible, non-transitory computer-readable media according to claim 22, wherein the inputs of ground truth rest or toss-turn states of the plurality of human subjects are based on corresponding depth images of the plurality of human subjects to identify the ground truth rest or toss-turn states.

24. The one or more tangible, non-transitory computer-readable media according to claim 23, wherein the ground truth toss-turn is found including operations of applying a fixed mask to the depth images and calculating the pixel-to-pixel difference in successive depth images, and then finding the energy in residual depth.

25. The one or more tangible, non-transitory computer-readable media according to claim 22, wherein training includes operations of leveraging cross-correlations between successive mmWave reflected signals to identify respective sleep versus disruption periods.

26. The one or more tangible, non-transitory computer-readable media according to claim 22, wherein operations further include further training the rest and toss-turn detection machine learning model to identify and separate two respective states of rest or toss-turn, and to estimate time gap between two adjacent resting periods.

27. The one or more tangible, non-transitory computer-readable media according to claim 22, wherein operations further include training including calculating envelopes from the reflected mmWave signals, and then predicting the binary states corresponding to rest and toss-turn.

28. The one or more tangible, non-transitory computer-readable media according to claim 27, further comprising operations of converting the envelopes with real-valued output between 0 to 1 to a discrete output of 0 and 1 as the predicted binary states corresponding to rest and toss-turn.

29. The one or more tangible, non-transitory computer-readable media according to claim 27, wherein predicting further comprises operations of using state transition and emission matrices and a Viterbi decoder to predict the binary states, corresponding to rest and toss-turn.

30. The one or more tangible, non-transitory computer-readable media according to claim 27, wherein calculating envelopes comprises further operations using a Hilbert Transformation, using the Root-Mean-Square (RMS) of cross-correlation amplitudes over N consecutive frames, wherein the number of N consecutive frames is in a range of about 3 to 50, for a corresponding range of RMS resolution of about 0.12 to 2 seconds of consecutive reflected signals, for envelope calculation.
